(19) United States
(12) Patent Application Publication
(10) Pub. No.: US 2026/0290523 A1
(43) Pub. Date: Sep. 24, 2026

(54) DATA ACQUISITION DEVICE

(71) Applicant: NTT DOCOMO, INC., Tokyo (JP)

(72) Inventors: Tomoko KAWASE, Tokyo (JP); Junya TAKIUE, Tokyo (JP); Kaho KATOU, Tokyo (JP); Nobuhiko NAKA, Tokyo (JP); Kei KIKUIRI, Tokyo (JP)

(73) Assignee: NTT DOCOMO, INC., Tokyo (JP)

(21) Appl. No.: 19/474,385

(22) PCT Filed: May 17, 2024
(86) PCT No.: PCT/JP2024/018353

§ 371 Date: Oct. 10, 2025

(30) Foreign Application Priority Data
May 18, 2023 (JP) 2023-082402

(51) Int. Cl. G16H 10/60; G16H 10/20; G16H 50/30

(52) U.S. Cl. CPC G16H 10/60; G16H 10/20; G16H 50/30

(57) ABSTRACT

This data acquisition device comprises: an information acquisition unit configured to acquire psychological reflection information of a user; an estimation unit configured to estimate the psychological state of the user by inputting the acquired psychological reflection information of the user into a psychological-state estimation model; a determination unit configured to determine whether or not it is necessary to acquire a correct label corresponding to the acquired psychological reflection information of the user; and a label creation unit configured to, when the determination unit determines that it is necessary to acquire the correct label, create the correct label corresponding to the psychological reflection information of the user based on the user's answer to a test presented to the user.

### TECHNICAL FIELD

The present disclosure relates to a data acquisition device.

### BACKGROUND ART

Patent Literature 1 discloses a technique for ascertaining and managing a mental state of an employee. This technique uses a learned model to determine whether or not an employee is at risk of mental illness on the basis of the employee's work situation.

### CITATION LIST

### Patent Literature

[Patent Literature 1] Japanese Unexamined Patent Publication No. 2022-37892

### SUMMARY OF INVENTION

### Technical Problem

When a learning model for estimating a user's psychological state from the user's information is constructed by machine learning, learning data (training data) corresponding to each psychological state is necessary. For example, when biological information and the like are provided from an information provider and a psychological state of the information provider is identified through a predetermined test and the like, learning data that is a set of user information and psychological state can be acquired. Although the learning data can be acquired from the information provider in each psychological state, it is difficult to uniformly acquire the learning data in each psychological state. In particular, the psychological state of people who cooperate in providing the learning data is often good, making it difficult to acquire learning data that corresponds to a poor psychological state.

The present disclosure has been made in view of the above problem, and an objective thereof is to provide a data acquisition device capable of acquiring necessary learning data.

### Solution to Problem

According to the present disclosure, a data acquisition device includes a psychological-state estimation model, an information acquisition unit, an estimation unit, a determination unit, a label creation unit, and a data storage unit. The psychological-state estimation model is trained using psychological reflection information and a correct label indicating a psychological state corresponding to the psychological reflection information as learning data. The information acquisition unit acquires the psychological reflection information of a user. The estimation unit estimates the psychological state of the user by inputting the psychological reflection information of the user acquired by the information acquisition unit into the psychological-state estimation model. The determination unit determines whether or not it is necessary to acquire the correct label corresponding to the acquired psychological reflection information of the user. The label creation unit creates the correct label corresponding to the psychological reflection information of the user on the basis of the user's answer to a test presented to the user when the determination unit determines that it is necessary to acquire the correct label. The data storage unit stores the psychological reflection information of the user and the correct label of the user as a data set.

In the above-described data acquisition device, the psychological-state estimation model estimates the psychological state of the user using the psychological reflection information of the user as input data. The data acquisition device can acquire the learning data by performing a predetermined test for the user and creating a correct label corresponding to the psychological reflection information of the user on the basis of the obtained answer to the test. Because such a data acquisition device has the determination unit that determines whether or not it is necessary to acquire the correct label corresponding to the acquired psychological reflection information of the user, it is possible to acquire the learning data as necessary.

### Advantageous Effects of Invention

According to the present disclosure, it is possible to provide a data acquisition device capable of acquiring necessary learning data.

### BRIEF DESCRIPTION OF DRAWINGS

FIG. 1 is a diagram showing a configuration of a data acquisition device according to an embodiment of the present disclosure.

FIG. 2 is a diagram showing a configuration of a determination unit as an example.

FIG. 3 is an explanatory flowchart of an operation of the determination unit as an example.

FIG. 4 is an explanatory flowchart of an operation of the determination unit as another example.

FIG. 5 is an explanatory flowchart of an operation of the determination unit as yet another example.

FIG. 6 is an explanatory flowchart of an operation of the determination unit as yet another example.

FIG. 7 is an explanatory flowchart of an operation of the determination unit as yet another example.

FIG. 8 is an explanatory flowchart of an operation of the determination unit as yet another example.

FIG. 9 is an explanatory flowchart of an example of an operation in the data acquisition device.

FIG. 10 is a diagram showing an example of a hardware configuration of the data acquisition device.

### DESCRIPTION OF EMBODIMENTS

Hereinafter, a data acquisition device according to the present disclosure will be described in detail with reference to the drawings. In the following description, the same reference signs are used for the same or corresponding elements, and redundant descriptions will be omitted.

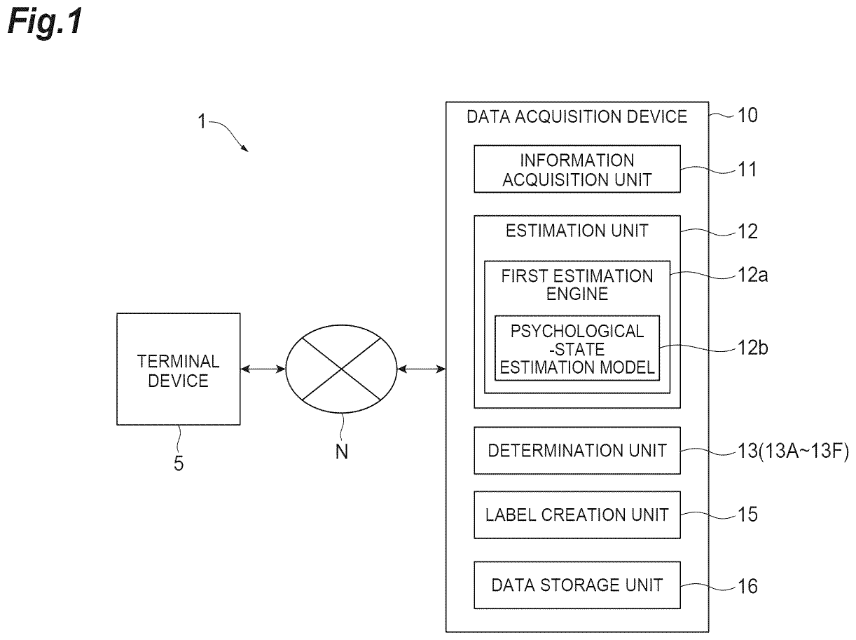

FIG. 1 is a diagram showing a configuration of a data acquisition system 1 including a data acquisition device 10 as an example. As an example, the data acquisition system 1 includes a terminal device 5 and a data acquisition device 10. For example, the data acquisition device 10 is configured to communicate with the terminal device 5 via a communication network N. Although only one terminal device 5 is shown in FIG. 1, the data acquisition device 10 may be connected to a plurality of terminal devices 5 simultaneously.

The terminal device 5 is, for example, a communication device that is operated by a user. The terminal device 5 receives an input, transmits received information to the data acquisition device 10, displays the information received from the data acquisition device 10, and the like. The terminal device 5 may be, for example, a portable terminal such as a highly functional portable phone (a smartphone), a portable phone, or a portable information terminal (PDA). In addition, the devices constituting the terminal device 5 are not to be interpreted in a restrictive manner.

The terminal device 5 can transmit psychological reflection information acquired from the user to the data acquisition device 10 in association with the user's identification information and the like stored in the terminal device 5. The psychological reflection information includes various types of data reflecting the user's psychological state. The psychological reflection information may include biological information of the user. The biological information is information indicating the state of a living body and can be acquired from the living body in accordance with a state at any given time. The biological information can be acquired by the terminal device 5. For example, the terminal device 5 may include a sensor for acquiring the biological information, may be connected to an external sensor, or may acquire data acquired by an external sensor.

As an example, the biological information may be at least one item of audio information, video information, and other detection information. The audio information may be sound data including speech uttered by the user. As an example, the audio information may be acquired by a microphone provided in the terminal device 5. The video information may be video data including an image captured of the user. The video information can include the user's facial expression, appearance of the eyes, gestures, and the like. The video information may be acquired by a camera provided in the terminal device 5. Other detection information serving as the biological information may be, for example, detection data related to the user's heartbeat, pulse, blood flow, body temperature, perspiration, respiration, brain waves, user movement, posture, and the like.

The data acquisition device 10 has an aspect of serving as an estimation device that estimates the psychological state of the user who uses the terminal device 5. The data acquisition device 10 also has an aspect of serving as an acquisition device to acquire learning data that can be used for the machine learning model for use in its estimation device. As shown in FIG. 1, as an example, the data acquisition device 10 has an information acquisition unit 11, an estimation unit 12, a determination unit 13, a label creation unit 15, and a data storage unit 16 as its functional units.

The information acquisition unit 11 acquires psychological reflection information of the user. As mentioned above, the user's psychological reflection information can be transmitted from the terminal device 5 used by the user to the data acquisition device 10. The information acquisition unit 11 can acquire the psychological reflection information of the user in association with the user's identification information. As an example, the information acquisition unit 11 may acquire the user's identification information, psychological reflection information, and date and time of reception (time information) in correspondence with each other. Moreover, the information acquisition unit 11 can acquire data indicating the attribute of the user in association with the user's identification information. The attribute may be a user-specific feature for classifying an individual without identifying the individual. The attribute may be, for example, gender, age, household composition, lifestyle, temperament, attendance information, or the like. Attribute data may be stored in the terminal device 5 in advance.

The estimation unit 12 estimates the psychological state of the user using the user's psychological reflection information acquired by the information acquisition unit 11 as input information. As shown in FIG. 1, as an example, the estimation unit 12 includes a first estimation engine 12a having a psychological-state estimation model 12b. The psychological-state estimation model 12b may be a machine learning model trained with the psychological reflection information and a correct label indicating a psychological state corresponding to the psychological reflection information as learning data. A machine learning framework may be, for example, any of regression, binary classification, anomaly detection, change detection, multi-class classification, and the like. In the present embodiment, an example in which the framework of the psychological-state estimation model 12b is regression will be described.

As an example, the learning data used for training the psychological-state estimation model 12b is configured to include a data set including data indicating psychological reflection information and a correct label obtained by quantifying the psychological state of the provider of the psychological reflection information. When the psychological reflection information is audio data, video data, or the like, the psychological reflection information may be acquired by the terminal device 5. The correct label is acquired on the basis of an answer to a predetermined test for the provider that is performed either simultaneously with or promptly after the acquisition of the psychological reflection information.

This test, for example, may include a plurality of questions to quantify a degree of psychological stress reaction. In other words, in this test, a measurement value obtained by quantifying a psychological state of a subject is decided. As an example, a question includes question text directed to the subject and a plurality of choices for the question text. After reading the question text, the subject can select his or her own answer from the choices. For example, a score may be assigned to each of the choices, and the measurement value may be decided on the basis of the score of the choice selected as the answer. The correct label constituting the learning data may be a numerical value obtained from a result of the test described above. In a learning model of the present embodiment, a test in which the measurement value increases as the stress increases is used. The data set consisting of psychological reflection information and correct-answer data may be associated with the provider's attribute and stored in the data storage unit 16.

A first estimation engine 12a of the estimation unit 12 acquires a numerical value (a measurement value) indicating the psychological state of the user outputted from the psychological-state estimation model 12b by inputting the psychological reflection information of the user into the learned psychological-state estimation model 12b. The first estimation engine 12a classifies the psychological state of the user as one of first and second categories. In other words, the first estimation engine 12a classifies the psychological state of the user as one of a poor state (the first category) and a good state (the second category) by classifying the acquired value by a first threshold value.

The estimation unit 12 estimates that the psychological state of the user is poor when the numerical value output from the psychological-state estimation model 12b is above a predetermined threshold value. Moreover, the estimation unit 12 estimates that the psychological state of the user is good when the numerical value output from the psychological-state estimation model 12b is less than the predetermined threshold value. The estimation unit 12 may transmit an estimation result to the terminal device 5 of the user. The transmitted estimation result may be only a category (a good state or a poor state) indicating a psychological state, or may include an estimated measurement value. The estimation unit 12 may store the estimated measurement value and category in association with identification information of the user. In other words, the estimation unit 12 may store the psychological state estimated in the past for each user.

The determination unit 13 determines whether or not it is necessary to acquire the correct label corresponding to the psychological reflection information acquired by the information acquisition unit 11. For example, to contribute to improving the accuracy of the psychological-state estimation model, the determination unit 13 determines whether or not it is necessary to acquire a correct label on the basis of content of psychological reflection information, a state of the psychological reflection information, a usage state of the user, a state of learning data in use, and the like. Details of the determination unit 13 will be described below.

The label creation unit 15 creates a correct label corresponding to the acquired psychological reflection information of the user when the determination unit 13 determines that the acquisition of the correct label is necessary. The correct label is created on the basis of the user's answer to a predetermined test presented to the user. The test is presented on a display of the terminal device 5 of the user when the determination unit 13 determines that the acquisition of the correct label is necessary. As an example, the label creation unit 15 may transmit the test to the terminal device 5 of the user whose correct label is required to be acquired. In addition, this test may have the same content as the test used to generate the learning data used to train the psychological-state estimation model.

The data storage unit 16 stores the psychological reflection information of the user acquired by the information acquisition unit 11 and the correct label created by the label creation unit 15 as a data set. This data may be associated with an attribute of the user and stored in data storage unit 16. The psychological-state estimation model 12b used by the estimation unit 12 can be updated with the data set newly stored in the data storage unit 16 as additional learning data.

Next, details of the determination unit 13 will be described. As an example, the determination unit 13 may be any one of a first determination unit 13A, a second determination unit 13B, a third determination unit 13C, a fourth determination unit 13D, a fifth determination unit 13E, and a sixth determination unit 13F that are shown in the following example.

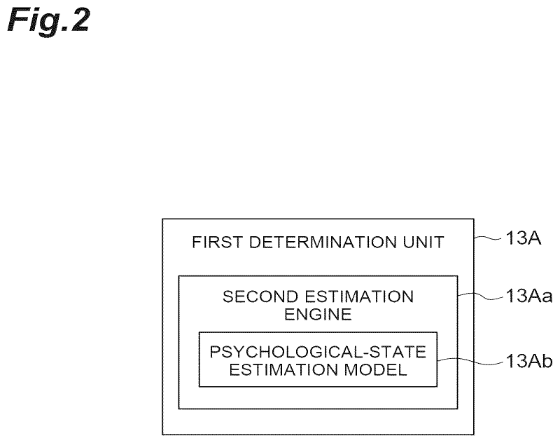

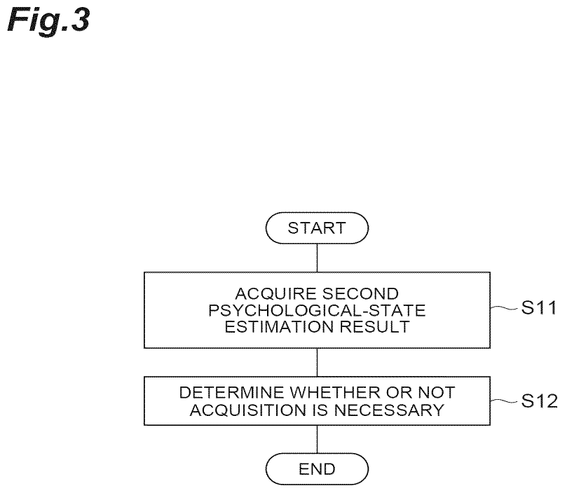

FIG. 2 is a block diagram showing a function of the first determination unit 13A. FIG. 3 is a flowchart showing an operation of the first determination unit 13A. The first determination unit 13A has a second estimation engine 13Aa with a psychological-state estimation model 13Ab. The psychological-state estimation model 13Ab included in the second estimation engine 13Aa may be the same learning model as the psychological-state estimation model 12b included in the estimation unit 12. In the first determination unit 13A, a second psychological-state estimation result is acquired by the second estimation engine 13Aa using the psychological-state estimation model 13Ab (step S11). In the second psychological-state estimation result, the proportion of determinations of a poor condition is higher than in the psychological state estimated by the estimation unit 12.

For example, the second estimation engine 13Aa can classify the psychological state of the user into third and fourth categories. The third category may be broader than the first category, which indicates that the user's psychological state is poor, and may encompass the first category. The fourth category may be narrower than the second category, which indicates that the user is in a good psychological state, and may be a category obtained by subtracting a portion overlapping the third category from the second category. The second estimation engine 13Aa classifies the user's psychological state as one of the third and fourth categories by classifying the measurement value estimated by the psychological-state estimation model 13Ab according to a second threshold value different from the first threshold value. In this case, the psychological reflection information indicating a measurement value near the threshold value for classifying the first and second categories is classified as the third category. That is, both users whose psychological state is poor in the first estimation engine 12a, and users with high measurement values among those whose psychological state is good, are classified as the third category. The first determination unit 13A determines whether or not it is necessary to acquire the correct label corresponding to the psychological reflection information of the user on the basis of the estimation result of the second estimation engine 13Aa (step S12). The first determination unit 13A determines that the acquisition of the correct label is necessary when the second estimation engine 13Aa estimates that the psychological reflection information is the third category.

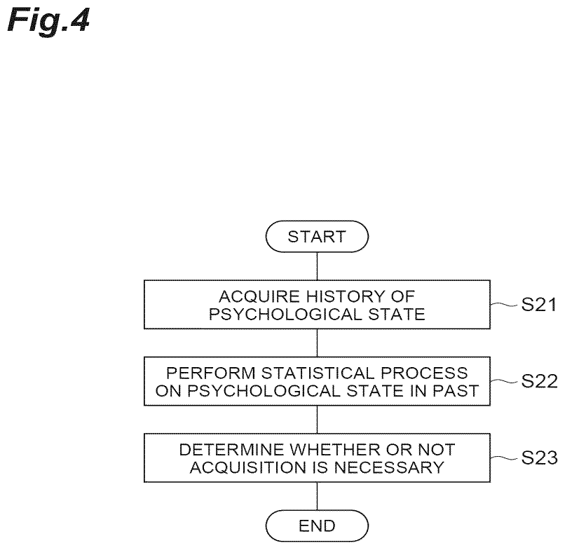

FIG. 4 is a flowchart showing an operation of the second determination unit 13B. The second determination unit 13B determines whether or not it is necessary to acquire the correct label corresponding to the acquired psychological reflection information of the user on the basis of the history of the user's psychological state. First, the second determination unit 13B acquires the history of the user's psychological state stored by the estimation unit 12 (step S21). For example, the second determination unit 13B may acquire a history of the user's psychological state acquired by the estimation unit 12 during a certain period of time in the past. Subsequently, the second determination unit 13B performs a statistical process on the user's past psychological states on the basis of the acquired psychological state history (step S22). Also, the second determination unit 13B determines whether or not it is necessary to acquire the correct label corresponding to the user's psychological reflection information on the basis of a result of the statistical process (step S23).

For example, the second determination unit 13B may acquire the proportion of the user's psychological state estimated to be a poor state on the basis of the acquired psychological state history. In this case, it may be determined that the acquisition of the correct label is necessary when the proportion of the user's psychological state estimated to be the poor state is greater than or equal to a predetermined threshold value. In addition, the second determination unit 13B may implement the determination by the first determination unit 13A when the proportion of the user's psychological state estimated to be the poor state is greater than or equal to the predetermined threshold value. In other words, the second determination unit 13B may determine that it is necessary to acquire the correct label when the proportion of the poor state in the user's past estimation results is greater than or equal to a predetermined proportion and a current estimation result of the first determination unit 13A is in the third category.

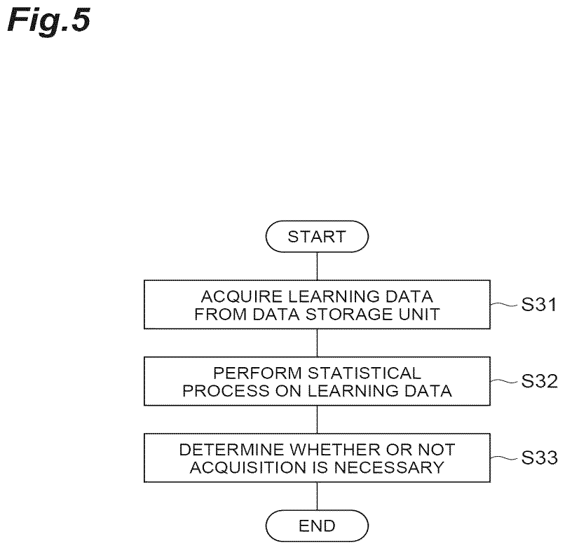

FIG. 5 is a flowchart showing an operation of the third determination unit 13C. The third determination unit 13C determines whether or not the acquisition of correct labels is necessary on the basis of the distribution of data stored in the data storage unit 16. For example, if a proportion of a specific category constituting the plurality of categories in the data stored in the data storage unit 16 is less than or equal to a predetermined value, when the psychological state of the user, estimated by inputting the psychological reflection information of the user into the psychological-state estimation model, corresponds to the specific category, the third determination unit 13C determines that it is necessary to acquire the correct label corresponding to the psychological reflection information of the user.

As an example, the third determination unit 13C first acquires the learning data stored in the data storage unit 16 (step S31). Subsequently, the third determination unit 13C performs a statistical process on the learning data (step S32) and determines whether or not it is necessary to acquire the correct label corresponding to the user's psychological reflection information on the basis of the result of the statistical process (step S33). For example, the third determination unit 13C calculates the proportion of a poor state on the basis of the category (a good or poor state) associated with the acquired learning data. Also, when the proportion of data indicating the poor state is less than or equal to a predetermined threshold value, it may be determined that the acquisition of the correct label is necessary. In addition, when the proportion of the poor state is less than or equal to the predetermined threshold value, the third determination unit 13C further implements the determination by the first determination unit 13A or the second determination unit 13B, and determines whether or not it is necessary to acquire the correct label on the basis of the determination result.

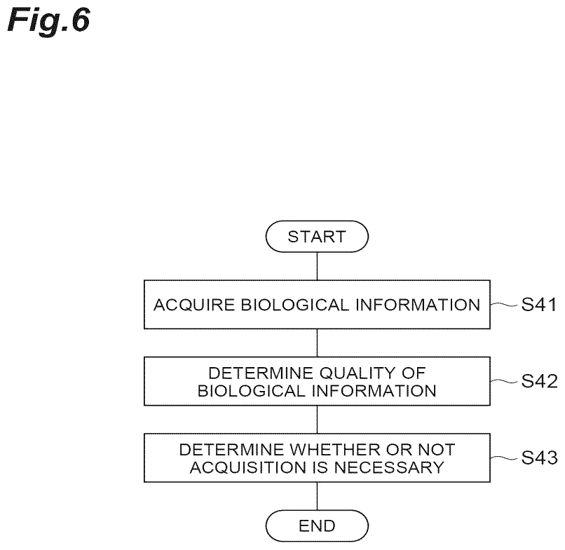

FIG. 6 is a flowchart showing an operation of the fourth determination unit 13D. The fourth determination unit 13D evaluates the quality of the user's biological information and determines whether or not it is necessary to acquire a correct label corresponding to the user's psychological reflection information in accordance with an evaluation result. First, the fourth determination unit 13D acquires the biological information constituting the user's psychological reflection information (step S41). Subsequently, the fourth determination unit 13D determines the quality of the biological information (step S42). The quality of the biological information may be an index of whether the data including the biological information can contribute to improving the learning accuracy of the psychological-state estimation model. For example, the quality of the biological information may be a signal-to-noise ratio in audio data, video data, and the like. The fourth determination unit 13D determines whether or not it is necessary to acquire a correct label on the basis of a result of determining the quality of the biological information (step S43). For example, the fourth determination unit 13D determines that the acquisition of the correct label is necessary when the quality of the biological information exceeds a predetermined criterion and determines that the acquisition of the correct label is unnecessary when the quality of the biological information is below the predetermined criterion.

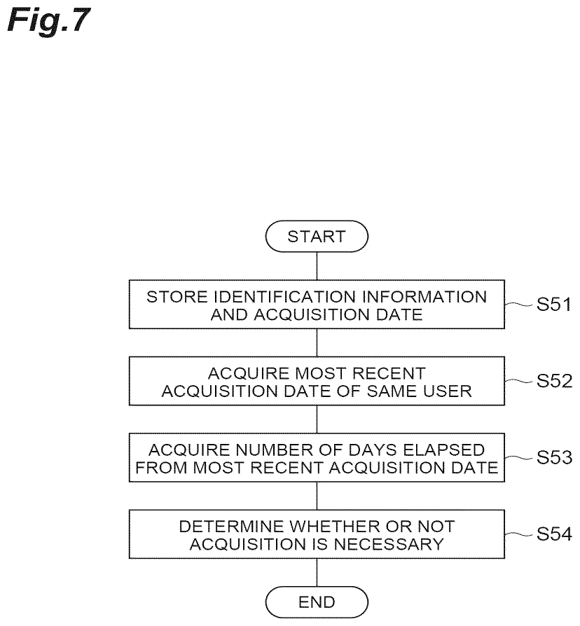

FIG. 7 is a flowchart showing an operation of the fifth determination unit 13E. The fifth determination unit 13E determines that the acquisition of the correct label corresponding to the user's psychological reflection information is necessary when a predetermined time has elapsed from the user's previous answer to the test for the acquisition of the correct label when the user's psychological reflection information has been acquired. For example, when the user's psychological reflection information is acquired by the information acquisition unit 11, the fifth determination unit 13E stores the user's identification information and the date of acquisition (step S51). Moreover, the fifth determination unit 13E stores whether or not there has been an answer to the test for acquiring the correct label in association with the acquisition date. The user's identification information and the acquisition date may be associated with the psychological reflection information and stored as a user history in the fifth determination unit 13E. When the user's psychological reflection information has been acquired, the fifth determination unit 13E refers to the user history and acquires a date of the most recent test answer of the user whose psychological reflection information has been acquired (step S52). Also, the fifth determination unit 13E calculates the number of days elapsed from the most recent acquisition date to the acquisition date of the user's psychological reflection information this time (step S53), and determines whether or not it is necessary to acquire a correct label in accordance with this number of elapsed days (step S54). For example, the fifth determination unit 13E determines that the acquisition of the correct label is necessary when the number of elapsed days is greater than or equal to a predetermined period, and determines that the acquisition of the correct label is unnecessary when the number of elapsed days is less than the predetermined period.

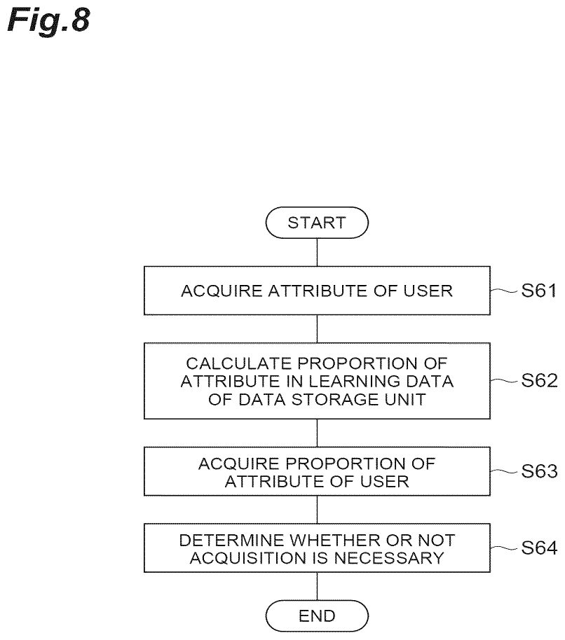

FIG. 8 is a flowchart showing an operation of the sixth determination unit 13F. The sixth determination unit 13F determines whether or not it is necessary to acquire a correct label on the basis of the distribution of data stored in the data storage unit 16. For example, the sixth determination unit 13F determines that the acquisition of the correct label corresponding to the user's psychological reflection information is necessary when the user's attribute is rare. First, the sixth determination unit 13F acquires the user's attribute on the basis of the user's identification information (step S61). The attribute may be associated with identification information and stored in the data storage unit 16. Subsequently, the sixth determination unit 13F calculates a proportion of each attribute in learning data (including additional learning data) stored in the data storage unit 16 (step S62). Subsequently, the sixth determination unit 13F acquires the proportion of the user's attribute within the learning data on the basis of the calculated proportion of each attribute (step S63). The sixth determination unit 13F determines whether or not it is necessary to acquire the correct label in accordance with the proportion of the user's attribute (step S64). For example, the sixth determination unit 13F may determine that the acquisition of the correct label is necessary when the proportion of the user's attribute is less than a predetermined threshold value, and may determine that the acquisition of the correct label is unnecessary when the proportion of the user's attribute is greater than or equal to the predetermined threshold value.

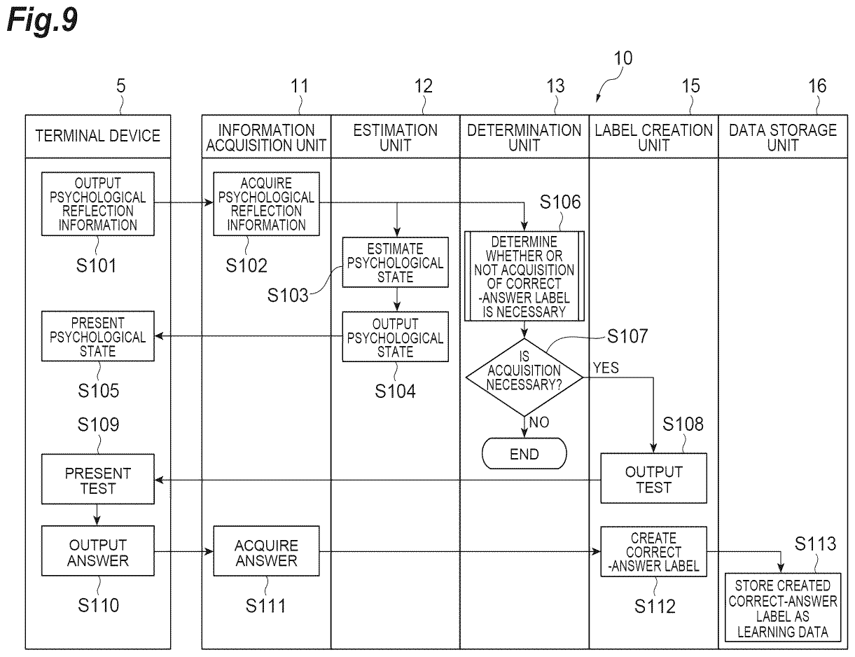

FIG. 9 is an explanatory flowchart of an example of an operation of the data acquisition system 1. As shown in FIG. 9, first, psychological reflection information is output from the terminal device 5 used by the user to the data acquisition device 10 (step S101). Subsequently, the information acquisition unit 11 acquires the psychological reflection information output from the terminal device 5 together with the user's identification information (step S102). The estimation unit 12 inputs the psychological reflection information acquired by the information acquisition unit 11 to the psychological-state estimation model 12b and acquires a result of estimating the psychological state of the user (step S103). The estimation unit 12 outputs the acquired estimation result to the terminal device 5 (step S104). The terminal device 5 acquires the estimation result output from the estimation unit 12 and displays the estimation result on a screen or the like (step S105). In addition, steps S104 and S105 may be omitted.

The determination unit 13 determines whether or not it is necessary to acquire a correct label with respect to the psychological reflection information transmitted by the user (steps S106 and S107). The determination unit 13 may be any one or a combination of the above-described determination units 13A to 13F. When it is determined that the acquisition of the correct label is unnecessary, the process of the data processing device may end. On the other hand, if it is determined that the acquisition of the correct label is necessary, the label creation unit 15 outputs the test to the terminal device 5 (step S108). In addition, when information about the test is stored in advance in the terminal device 5, the label creation unit 15 may output a command for displaying the test to the terminal device 5.

Subsequently, when the terminal device 5 displays the test on the screen (step S109), the user can provide an answer to the test. When the user provides the answer to the test, the terminal device 5 outputs an answer result to data acquisition device 10 (step S110). The information acquisition unit 11 acquires the answer result (step S111) and the correct label is created by the label creation unit 15 on the basis of the answer result (step S112). The correct label is a numerical value obtained by digitizing a degree of psychological stress reaction. The correct label is determined on the basis of the answer to the test. The created correct label is stored in the data storage unit 16 as the learning data together with the user's psychological reflection information and the user's attribute (step S113). The learning data newly stored in the data storage unit 16 can be used for updating (additionally training) the psychological-state estimation model. Moreover, the user's psychological state (good or poor) based on the created correct label may be transmitted to the terminal device 5.

As described above, as an example, the data acquisition device 10 includes the psychological-state estimation model 12b trained using psychological reflection information and a correct label indicating a psychological state corresponding to the psychological reflection information as learning data; the information acquisition unit 11 configured to acquire the psychological reflection information of a user; an estimation unit 12 configured to estimate the psychological state of the user by inputting the acquired psychological reflection information of the user into the psychological-state estimation model 12b; the determination unit 13 configured to determine whether or not it is necessary to acquire the correct label corresponding to the psychological reflection information of the user acquired by the information acquisition unit; the label creation unit 15 configured to create the correct label corresponding to the psychological reflection information of the user on the basis of the user's answer to a test presented to the user when the determination unit 13 determines that it is necessary to acquire the correct label; and the data storage unit 16 configured to store the psychological reflection information of the user and the correct label of the user as a data set.

When a learning model for estimating a user's psychological state is constructed by machine learning, learning data corresponding to the psychological state is necessary. In an example, learning data generated by the cooperation of the provider is used to construct a learning model. Although the learning data can be acquired from the provider in each psychological state, it is difficult to acquire learning data similarly in each psychological state. In particular, it is difficult to acquire learning data corresponding to a poor state because the psychological state of the provider who cooperates in generating learning data is rarely poor. In this case, the number of learning data items that correspond to a good state and the number of learning data items that correspond to a poor state may be biased, making it difficult to accurately estimate the psychological state.

In the above-described data acquisition device 10, the psychological-state estimation model estimates the user's psychological state using the user's psychological reflection information as input data. The data acquisition device 10 can acquire learning data by performing a predetermined test on the user and creating a correct label corresponding to the user's psychological reflection information on the basis of an obtained answer to the test. Because this data acquisition device 10 has a determination unit 13 that determines whether or not it is necessary to acquire the correct label corresponding to the acquired psychological reflection information of the user, it is possible to acquire the learning data as needed. In other words, when the estimated psychological state is poor, the acquisition of the correct label is determined to be necessary, thereby enabling the efficient acquisition of learning data corresponding to the poor state that is not easily acquired. Moreover, when the acquisition of the correct label is unnecessary, because an answer to the test from the user is not required, the excessive burden on the user is suppressed.

As an example, the psychological-state estimation model may be updated using the data stored in the data storage unit 16 as the learning data. In this configuration, the psychological-state estimation model can be updated in accordance with the determination criterion of the determination unit 13 because the learning data including psychological reflection information that is determined to be necessary by the determination unit 13 is reflected in the psychological-state estimation model.

As an example, the determination unit 13 may determine whether or not it is necessary to acquire the correct label on the basis of a distribution of the data stored in the data storage unit 16. In this configuration, for example, relatively insufficient learning data can be efficiently acquired in accordance with a distribution of learning data stored in the data storage unit 16.

In an example, the correct label constituting the learning data can be classified as one of a plurality of categories. The determination unit 13 may determine that it is necessary to acquire the correct label corresponding to the psychological reflection information of the user, if a proportion of a specific category among the plurality of categories in the data stored in the data storage unit 16 is less than or equal to a predetermined value, and when the psychological state of the user, estimated by inputting the psychological reflection information of the user into the psychological-state estimation model, corresponds to the specific category. In this configuration, when there is a category for which data collection is expected to be difficult in advance, the learning data corresponding to the category can be efficiently acquired.

In an example, the information acquisition unit 11 may acquire an attribute of the user, the data storage unit 16 may store the attribute together with the data, and the determination unit 13 may calculate a proportion of each attribute of the data stored in the data storage unit 16 and determine that it is necessary to acquire the correct label corresponding to the psychological reflection information of the user when the proportion of the attribute of the user acquired by the information acquisition unit 11 is less than or equal to a predetermined proportion. In this configuration, it is possible to efficiently acquire learning data corresponding to a user with a relatively small number of attributes. It is possible to reduce the burden on users with attributes for which sufficient learning data has been accumulated.

In an example, the estimation unit 12 may classify the psychological state of the user as one of first and second categories, and the determination unit 13 may classify the state of the user as one of a third category that is broader than the first category and including the first category and a fourth category obtained by subtracting the third category from the second category and determine that it is necessary to acquire the correct label corresponding to the psychological reflection information of the user when the state of the user is classified as the third category. In this configuration, psychological reflection information that could potentially be classified as the first category can be efficiently acquired among psychological reflection information items classified as the second category.

In an example, the estimation unit 12 may store the psychological state estimated in the past for each user, and the determination unit 13 may determine whether or not it is necessary to acquire the correct label corresponding to the acquired psychological reflection information of the user on the basis of a history of the psychological state of the user stored by the estimation unit 12. For example, if the user's psychological state history is found to have a high proportion of a poor state, reliable data can be obtained by acquiring the psychological reflection information when the user's psychological state is estimated to be poor.

In an example, the information acquisition unit 11 may acquire biological information of the user as the psychological reflection information, and the determination unit 13 may evaluate quality of the biological information of the user and determine that it is not necessary to acquire the correct label corresponding to the psychological reflection information of the user when the quality of the biological information of the user is below a predetermined criterion. In this configuration, because low-quality data is not acquired, the deterioration in the estimation accuracy of the psychological-state estimation model is suppressed.

As an example, the determination unit 13 may determine that it is necessary to acquire the correct label corresponding to the psychological reflection information of the user when a predetermined time has elapsed from acquisition of the user's previous answer to the test for acquiring the correct label when the psychological reflection information of the user has been acquired. When a long period of time has elapsed from the previous answer to the test to acquire the correct label, the reliability of the correct label is not necessarily considered high. This configuration prevents the reliability of correct labels from deteriorating. In addition, because the necessity of updating the correct label is determined on the basis of the elapsed time from the previous acquisition date of the correct label, the burden of an answer from the user for whom a certain period of time has not elapsed from the last acquisition date of the correct label can be reduced.

Although an embodiment is described in detail above with reference to the drawings, specific configurations are not limited to this embodiment. For example, the determination unit 13 may be a combination of the above-described determination units 13. For example, the determination unit 13 may be a combination of one of the first, second, and third determination units 13A, 13B, and 13C, and at least one of the fourth, fifth, and sixth determination units 13D, 13E, and 13F. In this case, if all of the determination units 13 of the combination determine that the acquisition of the correct model is necessary, the determination units 13 may determine that the acquisition of the correct model is necessary.

Although we have shown an example in which the user's psychological state is classified as one of two categories by the estimation unit and the like, the user's psychological state may be classified as one of more categories.

The block diagrams that have been used to describe the above embodiments show blocks in functional units. These functional blocks (components) may be implemented in arbitrary combinations of at least one of hardware and software. Also, the method for implementing each functional block is not particularly limited. That is, each functional block may be realized by one piece of apparatus that is physically or logically coupled, or may be realized by directly or indirectly connecting two or more physically or logically separate pieces of apparatus (for example, via wire, wireless, or the like) and using these plurality of pieces of apparatus. The functional blocks may be implemented by combining software into the apparatus described above or the plurality of apparatuses described above.

Functions include judgment, determination, decision, calculation, computation, processing, derivation, investigation, search, confirmation, reception, transmission, output, access, resolution, selection, designation, establishment, comparison, assumption, expectation, considering, broadcasting, notifying, communicating, forwarding, configuring, reconfiguring, allocating (mapping), assigning, and the like, but functions are by no means limited to these. For example, functional block (components) to implement a function of transmission may be referred to as a “transmitting section (transmitting unit),” a “transmitter,” and the like. The method for implementing each component is not particularly limited as described above.

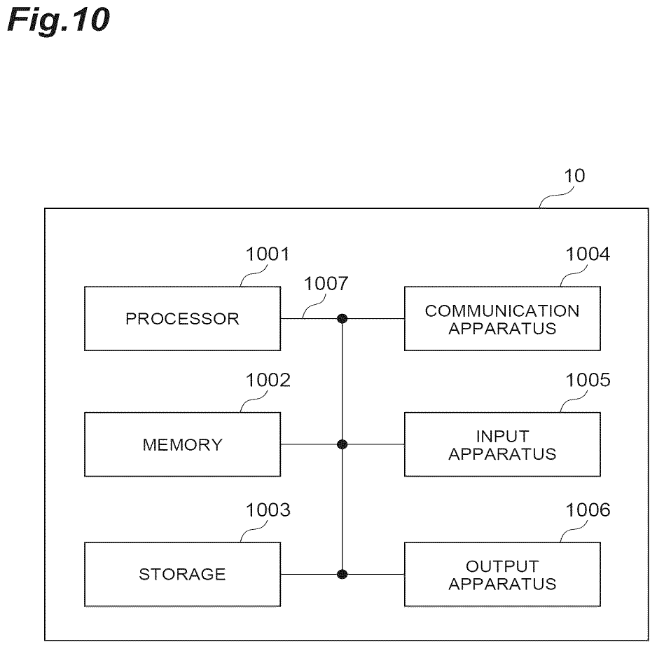

For example, the data acquisition device 10 according to one embodiment of the present disclosure may function as a computer that executes the processes of psychological-state estimation and data acquisition of the present disclosure. FIG. 10 is a diagram to show an example of a hardware structure of the data acquisition device 10 according to one embodiment. Physically, the above-described data acquisition device 10 may each be formed as a computer apparatus that includes a processor 1001, a memory 1002, a storage 1003, a communication apparatus 1004, an input apparatus 1005, an output apparatus 1006, a bus 1007, and so on.

Note that in the present disclosure, the words such as an apparatus, a circuit, a device, a section, a unit, and so on can be interchangeably interpreted. The hardware structure of the information acquisition unit 11, the estimation unit 12, the determination unit 13, the label creation unit 15, and the data storage unit 16 may be configured to include one or more of apparatuses shown in the drawings, or may be configured not to include part of apparatuses.

Each function of the data acquisition device is implemented, for example, by allowing certain software (programs) to be read on hardware such as the processor 1001 and the memory 1002, and by allowing the processor 1001 to perform calculations to control communication via the communication apparatus 1004 and control at least one of reading and writing of data in the memory 1002 and the storage 1003.

The processor 1001 controls the whole computer by, for example, running an operating system. The processor 1001 may be configured with a central processing unit (CPU), which includes interfaces with peripheral apparatus, control apparatus, computing apparatus, a register, and so on. For example, at least part of the above-described the estimation unit 12, the determination unit 13, the label creation unit 15 and so on may be implemented by the processor 1001.

Furthermore, the processor 1001 reads programs (program codes), software modules, data, and so on from at least one of the storage 1003 and the communication apparatus 1004, into the memory 1002, and executes various processes according to these. As for the programs, programs to allow computers to execute at least part of the operations of the above-described embodiments are used. For example, the estimation unit 12, the determination unit 13, the label creation unit 15 and so on may be implemented by control programs that are stored in the memory 1002 and that operate on the processor 1001, and other functional blocks may be implemented likewise. The various processes have been described to be performed by a single processor 1001. However, the processes may be performed by two or more processors 1001 simultaneously or sequentially. The processor 1001 may be implemented by one or more chips. It should be noted that the program may be transmitted from a network via a telecommunication line.

The memory 1002 is a computer-readable recording medium, and may be constituted with, for example, at least one of a Read Only Memory (ROM), an Erasable Programmable ROM (EPROM), an Electrically EPROM (EEPROM), a Random Access Memory (RAM), and other appropriate storage media. The memory 1002 may be referred to as a “register,” a “cache,” a “main memory (primary storage apparatus)” and so on. The memory 1002 can store executable programs (program codes), software modules, and the like for implementing the data acquisition method according to one embodiment of the present disclosure.

The storage 1003 is a computer-readable recording medium, and may be constituted with, for example, at least one of a flexible disk, a floppy (registered trademark) disk, a magneto-optical disk (for example, a compact disc (Compact Disc ROM (CD-ROM) and so on), a digital versatile disc, a Blu-ray (registered trademark) disk), a removable disk, a hard disk drive, a smart card, a flash memory device (for example, a card, a stick, and a key drive), a magnetic stripe, a database, a server, and other appropriate storage media. The storage 1003 may be referred to as “auxiliary storage apparatus.” The above recording medium may be a database including the memory 1002 and/or the storage 1003, a server, or any other appropriate medium.

The communication apparatus 1004 is hardware (transmitting/receiving device) for allowing inter-computer communication via at least one of wired and wireless networks, and may be referred to as, for example, a “network device,” a “network controller,” a “network card,” a “communication module,” and so on. The communication apparatus 1004 may be configured to include a high frequency switch, a duplexer, a filter, a frequency synthesizer, and so on in order to realize, for example, at least one of frequency division duplex (FDD) and time division duplex (TDD). For example, the information acquisition unit 11, and the like, may be implemented by the communication device 1004.

The input apparatus 1005 is an input device that receives input from the outside (for example, a keyboard, a mouse, a microphone, a switch, a button, a sensor, and so on). The output apparatus 1006 is an output device that allows sending output to the outside (for example, a display, a speaker, a Light Emitting Diode (LED) lamp, and so on). Note that the input apparatus 1005 and the output apparatus 1006 may be provided in an integrated structure (for example, a touch panel).

Furthermore, these types of apparatus, including the processor 1001, the memory 1002, and others, are connected by a bus 1007 for communicating information. The bus 1007 may be formed with a single bus, or may be formed with buses that vary between pieces of apparatus.

Also, the data acquisition device may be structured to include hardware such as a microprocessor, a digital signal processor (DSP), an Application Specific Integrated Circuit (ASIC), a Programmable Logic Device (PLD), a Field Programmable Gate Array (FPGA), and so on, and part or all of the functional blocks may be implemented by the hardware. For example, the processor 1001 may be implemented with at least one of these pieces of hardware.

Notification of information is by no means limited to the aspects/embodiments described in the present disclosure, and other methods may be used as well. For example, notification of information in the present disclosure may be implemented by using physical layer signaling (for example, downlink control information (DCI), uplink control information (UCI)), higher layer signaling (for example, Radio Resource Control (RRC) signaling, broadcast information (master information block (MIB), system information block (SIB), and so on), Medium Access Control (MAC) signaling), and other signals or combinations of these. Also, RRC signaling may be referred to as an “RRC message,” and can be, for example, an RRC connection setup message, an RRC connection reconfiguration message, and so on.

The order of processes, sequences, flowcharts, and so on that have been used to describe the aspects/embodiments in the present disclosure may be re-ordered as long as inconsistencies do not arise. For example, although various methods have been illustrated in the present disclosure with various components of steps in exemplary orders, the specific orders that are illustrated herein are by no means limiting.

The input or output information may be stored in a specific location (e.g., memory) or managed using management tables. The input or output information may be overwritten, updated, or added. The information that has been output may be deleted. The information that has been input may be transmitted to another apparatus.

A decision or a determination in an embodiment of the present invention may be realized by a value (0 or 1) represented by one bit, by a boolean value (true or false), or by comparison of numerical values (e.g., comparison with a predetermined value).

Each aspect/embodiment described in the present specification may be used independently, may be used in combination, or may be used by switching according to operations. Further, notification (transmission/reporting) of predetermined information (e.g., notification (transmission/reporting) of “X”) is not limited to an explicit notification (transmission/reporting), and may be performed by an implicit notification (transmission/reporting) (e.g., by not performing notification (transmission/reporting) of the predetermined information).

As described above, the present invention has been described in detail. It is apparent to a person skilled in the art that the present invention is not limited to one or more embodiments of the present invention described in the present specification. Modifications, alternatives, replacements, etc., of the present invention may be possible without departing from the subject matter and the scope of the present invention defined by the descriptions of claims. Therefore, the descriptions of the present specification are for illustrative purposes only, and are not intended to be limitations to the present invention.

Software should be broadly interpreted to mean, whether referred to as software, firmware, middle-ware, microcode, hardware description language, or any other name, instructions, instruction sets, codes, code segments, program codes, programs, subprograms, software modules, applications, software applications, software packages, routines, subroutines, objects, executable files, executable threads, procedures, functions, and the like.

Further, software, instructions, information, and the like may be transmitted and received via a transmission medium. For example, in the case where software is transmitted from a website, server, or other remote source using at least one of wired line technologies (such as coaxial cable, fiber optic cable, twisted pair, digital subscriber line (DSL), etc.) or wireless technologies (infrared, microwave, etc.), at least one of these wired line technologies or wireless technologies is included within the definition of the transmission medium.

Information, a signal, or the like, described in the present specification may be represented by using any one of various different technologies. For example, data, an instruction, a command, information, a signal, a bit, a symbol, a chip, or the like, described throughout the present application, may be represented by a voltage, an electric current, electromagnetic waves, magnetic fields, a magnetic particle, optical fields, a photon, or a combination thereof.

It should be noted that a term used in the present specification and/or a term required for understanding of the present specification may be replaced by a term having the same or similar meaning.

Further, the information, parameters, and the like, described in the present disclosure may be expressed using absolute values, relative values from predetermined values, or they may be expressed using corresponding different information. For example, a radio resource may be what is indicated by an index.

As used herein, the term “determining” may encompasses a wide variety of actions. For example, “determining” may be regarded as judging, calculating, computing, processing, deriving, investigating, looking up (e.g., looking up in a table, a database or another data structure), ascertaining and the like. Also, “determining” may be regarded as receiving (e.g., receiving information), transmitting (e.g., transmitting information), inputting, outputting, accessing (e.g., accessing data in a memory) and the like. Also, “determining” may be regarded as resolving, selecting, choosing, establishing and the like. That is, “determining” may be regarded as a certain type of action related to determining.

The term “connected” or “coupled” or any variation thereof means any direct or indirect connection or connection between two or more elements and may include the presence of one or more intermediate elements between the two elements “connected” or “coupled” with each other. The coupling or connection between the elements may be physical, logical, or a combination thereof. For example, “connection” may be read as “access”. As used in the present disclosure, the two elements may be thought of as being “connected” or “coupled” to each other using at least one of the one or more wires, cables, or printed electrical connections and, as a number of non-limiting and non-inclusive examples, electromagnetic energy having wavelengths in the radio frequency region, the microwave region, and the light (both visible and invisible) region.

The phrase “based on” (or “on the basis of”) as used in the present disclosure does not mean “based only on” (or “only on the basis of”), unless otherwise specified. In other words, the phrase “based on” (or “on the basis of”) means both “based only on” and “based at least on” (“only on the basis of” and “at least on the basis of”).

Reference to elements with designations such as “first,” “second,” and so on as used in the present disclosure does not generally limit the quantity or order of these elements. These designations may be used in the present disclosure only for convenience, as a method for distinguishing between two or more elements. Thus, reference to the first and second elements does not imply that only two elements may be employed, or that the first element must precede the second element in some way.

In the case where the terms “include”, “including” and variations thereof are used in the present disclosure, these terms are intended to be comprehensive in the same way as the term “comprising”. Further, the term “or” used in the present specification is not intended to be an “exclusive or”.

In the present disclosure, where an article is added by translation, for example “a”, “an”, and “the”, the disclosure may include that the noun following these articles is plural.

In this disclosure, the term “A and B are different” may mean “A and B are different from each other.” It should be noted that the term “A and B are different” may mean “A and B are different from C.” Terms such as “separated” or “combined” may be interpreted in the same way as the above-described “different”.

The estimation device of the present disclosure has the following configurations.

[1] A data acquisition device comprising:
a psychological-state estimation model trained using psychological reflection information and a correct label indicating a psychological state corresponding to the psychological reflection information as learning data;
an information acquisition unit configured to acquire the psychological reflection information of a user;
an estimation unit configured to estimate the psychological state of the user by inputting the psychological reflection information of the user acquired by the information acquisition unit into the psychological-state estimation model;
a determination unit configured to determine whether or not it is necessary to acquire the correct label corresponding to the psychological reflection information of the user acquired by the information acquisition unit;
a label creation unit configured to create the correct label corresponding to the psychological reflection information of the user on the basis of the user's answer to a test presented to the user when the determination unit determines that it is necessary to acquire the correct label; and
a data storage unit configured to store the psychological reflection information of the user and the correct label of the user as a data set.

[2] The data acquisition device according to [1], wherein the psychological-state estimation model is updated using the data stored in the data storage unit as the learning data.

[3] The data acquisition device according to [1] or [2], wherein the determination unit determines whether or not it is necessary to acquire the correct label on the basis of a distribution of the data stored in the data storage unit.

[4] The data acquisition device according to [3],
wherein the correct label constituting the learning data can be classified as one of a plurality of categories, and
wherein the determination unit is configured to determine that it is necessary to acquire the correct label corresponding to the psychological reflection information of the user, if a proportion of a specific category among the plurality of categories in the data stored in the data storage unit is less than or equal to a predetermined value, and when the psychological state of the user, estimated by inputting the psychological reflection information of the user into the psychological-state estimation model, corresponds to the specific category.

[5] The data acquisition device according to [3],
wherein the information acquisition unit acquires an attribute of the user,
wherein the data storage unit stores the attribute together with the data, and
wherein the determination unit calculates a proportion of each attribute of the data stored in the data storage unit and determines that it is necessary to acquire the correct label corresponding to the psychological reflection information of the user when the proportion of the attribute of the user acquired by the information acquisition unit is less than or equal to a predetermined proportion.

[6] The data acquisition device according to any one of [1] to [5],
wherein the estimation unit classifies the psychological state of the user as one of first and second categories, and
wherein the determination unit classifies the state of the user as one of a third category that is broader than the first category and including the first category and a fourth category obtained by subtracting the third category from the second category and determines that it is necessary to acquire the correct label corresponding to the psychological reflection information of the user when the state of the user is classified as the third category.

[7] The data acquisition device according to any one of [1] to [6],
wherein the estimation unit stores the psychological state estimated in the past for each user, and
wherein the determination unit determines whether or not it is necessary to acquire the correct label corresponding to the acquired psychological reflection information of the user on the basis of a history of the psychological state of the user stored by the estimation unit.

[8] The data acquisition device according to any one of [1] to [7], wherein the information acquisition unit acquires biological information of the user as the psychological reflection information, and wherein the determination unit evaluates a quality of the biological information of the user and determines that it is not necessary to acquire the correct label corresponding to the psychological reflection information of the user when the quality of the biological information of the user is below a predetermined criterion.

[9] The data acquisition device according to any one of [1] to [8], wherein the determination unit determines that it is necessary to acquire the correct label corresponding to the psychological reflection information of the user when a predetermined time has elapsed from acquisition of the user's previous answer to the test when the psychological reflection information of the user has been acquired.

### REFERENCE SIGNS LIST

10 Data acquisition device, 11 Information acquisition unit, 12 Estimation unit, 13 Determination unit, 15 Label creation unit, 16 Data storage unit, 1001 Processor, 1002 Memory, 1003 Storage, 1004 Communication apparatus, 1005 Input apparatus, 1006 Output apparatus, 1007 Bus

1. A data acquisition device comprising:
a psychological-state estimation model trained using psychological reflection information and a correct label indicating a psychological state corresponding to the psychological reflection information as learning data;
an information acquisition unit configured to acquire the psychological reflection information of a user;
an estimation unit configured to estimate the psychological state of the user by inputting the psychological reflection information of the user acquired by the information acquisition unit into the psychological-state estimation model;
a determination unit configured to determine whether or not it is necessary to acquire the correct label corresponding to the psychological reflection information of the user acquired by the information acquisition unit;
a label creation unit configured to create the correct label corresponding to the psychological reflection information of the user based on the user's answer to a test presented to the user when the determination unit determines that it is necessary to acquire the correct label; and
a data storage unit configured to store the psychological reflection information of the user and the correct label of the user as a data set.

2. The data acquisition device according to claim 1, wherein the psychological-state estimation model is updated using the data stored in the data storage unit as the learning data.

3. The data acquisition device according to claim 1, wherein the determination unit determines whether or not it is necessary to acquire the correct label based on a distribution of the data stored in the data storage unit.

4. The data acquisition device according to claim 3,
wherein the correct label constituting the learning data can be classified as one of a plurality of categories, and
wherein the determination unit is configured to determine that it is necessary to acquire the correct label corresponding to the psychological reflection information of the user, if a proportion of a specific category among the plurality of categories in the data stored in the data storage unit is less than or equal to a predetermined value, and when the psychological state of the user, estimated by inputting the psychological reflection information of the user into the psychological-state estimation model, corresponds to the specific category.

5. The data acquisition device according to claim 3,
wherein the information acquisition unit acquires an attribute of the user,
wherein the data storage unit stores the attribute together with the data, and
wherein the determination unit calculates a proportion of each attribute of the data stored in the data storage unit and determines that it is necessary to acquire the correct label corresponding to the psychological reflection information of the user when the proportion of the attribute of the user acquired by the information acquisition unit is less than or equal to a predetermined proportion.

6. The data acquisition device according to claim 1,
wherein the estimation unit classifies the psychological state of the user as one of first and second categories, and
wherein the determination unit classifies the state of the user as one of a third category that is broader than the first category and including the first category and a fourth category obtained by subtracting the third category from the second category and determines that it is necessary to acquire the correct label corresponding to the psychological reflection information of the user when the state of the user is classified as the third category.

7. The data acquisition device according to claim 1,
wherein the estimation unit stores the psychological state estimated in the past for each user, and
wherein the determination unit determines whether or not it is necessary to acquire the correct label corresponding to the acquired psychological reflection information of the user based on a history of the psychological state of the user stored by the estimation unit.

8. The data acquisition device according to claim 1,
wherein the information acquisition unit acquires biological information of the user as the psychological reflection information, and
wherein the determination unit evaluates a quality of the biological information of the user and determines that it is not necessary to acquire the correct label corresponding to the psychological reflection information of the user when the quality of the biological information of the user is below a predetermined criterion.

9. The data acquisition device according to claim 1, wherein the determination unit determines that it is necessary to acquire the correct label corresponding to the psychological reflection information of the user when a predetermined time has elapsed from acquisition of the user's previous answer to the test when the psychological reflection information of the user has been acquired.
